(19) United States
(12) Patent Application Publication
(10) Pub. No.: US 2020/0164037 A1
(43) Pub. Date: May 28, 2020

(54) DERMOCOSMETIC COMPOSITION CONTAINING SYNTHETIC PLATELET-LIKE MOLECULES TO TREAT SKIN AGING AND STIMULATE HAIR GROWTH

(71) Applicant: LABO COSPROPHAR AG, Basel (CH)

(72) Inventors: Daniela Montanari, Albignasego (PD) (IT); Manuela Guglielmo, Vigonovo (IT)

(73) Assignee: LABO COSPROPHAR AG, Basel (CH)

(21) Appl. No.: 16/200,751

(22) Filed: Nov. 27, 2018

(51) Int. Cl. A61K 38/18; A61K 8/04; A61Q 19/08; A61K 8/06; A61Q 19/00; A61Q 7/00; A61K 8/64; A61K 38/30

(52) U.S. Cl. CPC A61K 38/1858; A61K 8/042; A61Q 19/08; A61K 8/06; A61Q 19/00; A61K 38/1841; A61K 8/64; A61K 38/1825; A61K 38/30; A61K 38/1866; A61K 38/1808; A61Q 7/00

(57) ABSTRACT

The present invention relates to a dermocosmetic composition for topical use comprising a combination of selected synthetic platelet-like molecules which find application in the trichological field in preventing or treating the thinning of the scalp and in the aesthetic field in preventing or treating the signs of skin aging such as roughness and loss of elasticity of the skin.

The present invention relates to a dermocosmetic composition containing synthetic platelet-like molecules to treat skin aging and to stimulate hair growth.

The present invention originates in the field of preparations for external cosmetic use and for trichological use.

In particular, the invention regards a composition and a dermocosmetic method of treatment with topical application based on Platelet Like Molecules suitable for improving the appearance of the skin, reducing the signs of skin aging and stimulating the physiological hair growth.

### Technique Note

The aesthetic and regenerative medicine develops techniques and methods derived from other branches of medicine to remedy aesthetic problems, such as skin aging, thinning hair on scalp and forms of baldness.

The skin aging is due to a series of concomitant factors among which the gradual decline of its functions over time (intrinsic aging) and the damage accumulation caused by environmental factors (extrinsic aging) that include smoke, exposure to chemical agents and UVB radiation. The role of growth factors in the rejuvenation of aged skin was highlighted in 2011 in an in vitro study conducted on fibroblasts (Kim D H, Lee Y).

Currently there are numerous cosmetic preparations on the market containing biologically active substances such as hyaluronic acid and polynucleotides which are applied both in cosmetics and in aesthetic medicine.

Recently, some methodologies that have found the main application in the medical-surgical field and which are based on the nutritive and regenerative blood properties have been transferred to the aesthetic medicine field.

One of these techniques utilized in the aesthetic medicine field involves the execution of subcutaneous microinjections of Platelet Rich Plasma (PRP).

Platelet Rich Plasma (PRP) is a liquid substance consisting of autologous blood plasma that is enriched with platelets after a series of centrifugations after venous sampling. The PRP, being a concentrated source of autologous platelets, contains different growth factors present in the platelets alpha-granules that turned out to be suitable for stimulating the healing of bones and tissues.

The PRP found application in various medical fields, such as in dermatology, orthopedics and dentistry. Currently, PRP is frequently applied in some cosmetic medicine treatments as a tool of stimulating tissue regeneration.

As is known, platelets are the basis of the tissue repair mechanism and hemostasis. In the presence of a wound they form the platelet cap and induce the release of substances that stimulate the reparative tissues mechanisms and the reactivity of other cells involved in the healing mechanism. Furthermore, platelets promote regeneration and modulate the inflammatory response by releasing growth factors, cytokines and other regulatory molecules.

The skin bioregeneration is a rejuvenation technique of the face and neck skin that consists in the stimulation and activation of the skin fibroblasts by infiltration into derm thickness of PRP in gel form.

Bioregeneration with PRP allows to restore the best skin life conditions, leading to an improvement in skin aesthetics and optimization of skin physiological parameters. The skin PRP administration induces and accelerates the regeneration process and the tissue reconstruction by: Stimulation of new collagen production; Remodeling of collagen fibers; Mesenchymal stem cells division and differentiation induction and angiogenesis stimulation (development of new blood vessels).

The skin quality and texture are therefore improved thanks to the increase in the collagen, elastin, fibronectin, and hyaluronic acid expression (Ebisawa et al., 2009).

The PRP skin treatment can be expected both in young subjects of 28-30 years as prevention, and in mature skin as skin aging treatment. To obtain an appreciable result, 3 sessions are required, spaced by a month in which direct injection of PRP into the dermis is foreseen.

This procedure can be performed, depending on the case, in different body regions such as the face, neck, decolleté, back of the hands, abdomen, inner portion of the arms and thighs. Another field of application of the PRP concerns the trichological one, in the treatment of baldness. The PRP modulates the hair bulb cycle and stimulates hair growth. The growth factors contained in it act on specific hair system cellular targets such as the dermal papilla, the bulge, the follicle root sheaths and the bulb, stimulating cell proliferation, differentiation and survival. PRP prolongs the anagen phase, reduces the telogen phase and delays the involution of the follicle. Furthermore, it has angiogenic potential, i.e. it stimulates the new blood capillaries formation on the scalp.

For the PRP preparation there are several protocols, but in general the plasma taken from an individual is placed in test tubes and concentrated in platelets in order to reach 3-5 times the physiological value. Generally, the tubes containing the blood are placed in a laboratory centrifuge and centrifuged. If the centrifugation conditions are inadequate, you risk sedimenting the platelets and obtaining a plasma, that is a PRP, poor in platelets. A special centrifuge suitable for this purpose should therefore be used with suitable acceleration, speed and deceleration modes. Without ever coming into contact with the environment the platelet-poor plasma is aspirated and discarded. Then the platelet-rich plasma is aspirated with the syringe which will then be used for treatment.

The PRP is then injected subcutaneously directly into the area of the face or scalp of the individual needing treatment, where the growth factors contained in the platelets can stimulate the regeneration of skin tissues or the growth of new hair. The platelets present in the PRP are activated with thrombin and calcium chloride or calcium gluconate in order to induce the release of growth factors from the alpha-granules.

In the case of facial skin treatment, a modest amount of blood of 20 cc is taken from the patient. In a special container the blood material is centrifuged using a special centrifuge dedicated to the purpose. Then proceed to infiltrate the plasma rich in platelets previously isolated according to a chessboard pattern.

In the case of scalp treatment, the amount of venous blood taken can vary from 20 cc to 40 cc depending on the extent of the area to be treated. In cases of low degree of hair thinning, 20 cc of blood is sufficient. In cases of extensive thinning on all or most of the scalp, 40 cc of venous blood may be required. Generally, from 20 cc of blood, about 3 cc of PRP are obtained with a high platelet concentration, while about 6 cc of PRP with high platelet concentration are obtained from 40 cc of blood.

One of the main limitations of the aesthetic medicine techniques that use the PRP is the invasiveness of the method which involves a series of subcutaneous injections of the platelet concentrate. Generally, these injections are performed by specialized medical or nursing staff.

Furthermore, the subcutaneous treatment with PRP is particularly painful since a discrete amount of liquid, injected at 0.05/0.1 ml/cm², is injected in a limited area. To limit this problem in many cases, local skin anesthesia applications are used.

Other methods in the field of aesthetic medicine that use the PRP provide for the activation of the platelets only after their injection, using the microneeding technique. Typically, in order to achieve this technique, a medical device is used which provides a pen with single-use micro-needles which pierce the skin vertically, thus substantially improving the absorption of the associated products.

The depth of action is adjustable according to the type of skin to be treated, the treatment area and the objective of the application, and varies from <0.5 mm to a maximum of 2.5 mm. The skin micropores induced by needling encourage and reinforce the physiological mechanisms of collagen production, elastin and other natural substances.

However, this technique has the further disadvantage of having a low compliance with the patient and being of complicated implementation.

In addition, aesthetic medicine treatments with PRP involve high costs especially when the therapy involves a cycle with a plurality of sessions. Last but not least, aesthetic medicine treatments with microinjection of PRP not only cannot be carried out in a domestic environment but also require carrying out a plurality of sessions.

At present, therefore, the need is felt to have a treatment for aesthetic problems such as skin aging and hair thinning that is alternative to conventional treatments of aesthetic medicine.

One of the aims of the present invention therefore consists in providing a composition for external use for the treatment and prevention of skin aging and/or thinning of the scalp which provides results similar to those obtained with aesthetic medicine, without resorting to intradermal injections of regenerating substances.

Another object of the invention is to provide a dermocosmetic treatment method which is alternative to cosmetic medicine treatments based on the intradermal inoculation of PRP and which entails considerably lower execution costs.

A further object is to provide a dermocosmetic composition to be applied locally on the skin which causes a regeneration of the skin tissue and/or a stimulation of hair growth similar to that obtainable with aesthetic medicine treatments with PRP, without however providing for invasiveness and the typical costs of treatment with PRP.

### SUMMARY

The Applicant has now identified and selected a group of specific synthetic platelet-like molecules that applied through the skin or on the scalp, providing an activity of skin regeneration and/or development of the follicle, the bulb and the hair assimilable to those obtained with the aesthetic medicine techniques.

According to a first aspect a dermocosmetic composition for topical application is provided for the stimulation of the hair growth comprising synthetic platelet-like molecules (MPS):
Synthetic human acidic fibroblast growth factor or sh-Polypeptide-11
Synthetic human basic Fibroblast growth factor or sh-Polypeptide-1
Synthetic human keratinocyte Growth Factor or sh-Polypeptide-3
Synthetic human insulin like 1 Growth Factor or sh-Oligopeptide-2
Synthetic human insulin like 2 Growth factor sh-Polypeptide-31
Synthetic human vascular Endothelial Growth Factor or sh-Polypeptide-9
Synthetic Human Epidermal Growth Factor or sh-Oligopeptide-1 and a cosmetically acceptable vehicle.

### DETAILED DESCRIPTION OF THE INVENTION

Typically, the 7 active components of the composition of the invention are synthetic Platelet-like Molecules (MPS), of dermocosmetic use.

The 7 active components of the composition do not derive from human blood or biological fluids but are produced by a synthesis process.

Typically, the similar platelet molecules used in the scope of the invention are proteins obtained by transcription and translation of a fragment of recombinant DNA inserted into a recipient cell of bacteria or yeasts and subsequent bacterial fermentation. The abbreviation sh- before the INCI name of the seven molecules PS means synthetic human.

The composition containing the 7 Synthetic Platelet-like Molecules is applied topically on the scalp, without the need for subcutaneous injections.

It has been observed that the mixture of the seven growth factors previously identified, when applied to the scalp, acts synergistically stimulating the activity of the hair bulb and thus promoting the physiological growth of the hair in the treated individual.

One of the components of the invention composition is the Synthetic Human Acidic Fibroblast Growth Factor, INCI Name sh-Polypeptide-11, PM 17,200 dalton.

This component belongs to a family of platelet molecules active in mitogenesis, cell migration, differentiation, angiogenesis and wound healing. It is a substance that induces the proliferation and physical organization of endothelial cells in tubular structures with angiogenic effect and therefore promotes the formation of new blood capillaries. At the level of the hair follicles the receptor for this molecule has been identified in the dermal papilla and in the inner sheath of the follicle root.

Another composition component is represented by Synthetic Human Basic Fibroblast Growth Factor, INCI Name sh-Polypeptide-1. Preferably this substance has a PM of 17200 daltons. This substance stimulates the proliferation of fibroblasts and angiogenesis. The receptor for this molecule was determined in the cells of the piliferous matrix, i.e. the cells adjacent to the dermal papilla in the hair bulb and in the outer sheath of the follicle root.

The Applicant has verified that the topical application of Sh-Polypetide-1 promotes the induction of the anagen phase, stimulating the hair follicles growth phase.

A further composition component is represented by sh-Polypeptide-3 or Synthetic Human Keratinocyte Growth Factor, a substance produced by the skin following skin lesions which promotes healing. Preferably this substance has a PM 22,500 daltons. This component can also be synthesized from skin fibroblasts and induces the proliferation of a wide variety of epithelial cells, including epidermal keratinocytes and keratinocytes present in hair follicles and sebaceous glands. This component is a hair growth regulator and is typically produced in developing hair follicles and in follicle dermal papilla. It is also provided with a stimulating effect on the growth of hair follicles and on the in vivo differentiation of keratin pilifera.

An additional component is represented by Synthetic Human Insulin-Like 1 Growth Factor, with INCI Name sh-Oligopeptide-2. Preferably this substance has PM 7,600 daltons. It is a platelet molecule that activates tissue growth and plays a crucial role as a promoter of hair growth. Typically, it is also produced by the dermal papilla and acts on the keratinocytes of the hair bulbs stimulating their proliferation and thus promoting the growth of hair. It has been observed that it maintains the anagen phase of the hair follicle and strengthens the hair shaft. Furthermore, this substance increases the energy supply to the follicles and stimulates the blood circulation of the scalp. It also has an anti-apoptotic effect, i.e. it reduces cell death.

An additional component is represented by Synthetic Human Insulin-Like 2 Growth Factor, or sh-Polipeptide-31. Preferably this substance has PM 7,600 daltons. It is an important molecule to maintain a physiological cellular metabolism and regulates many biochemical functions related to organ growth. It is a platelet-like molecule provided with a stimulating activity on the proliferation of keratinocytes. It has been observed that it stimulates the growth of hair follicles and plays an important role as a regulator of the hair growth cycle preventing the entry into catagen of the follicle. Furthermore, it stimulates the hair follicles to produce stems with a physiological dimension.

An additional component is represented by Synthetic Human Vascular Endothelial Growth Factor, INCI Name sh-Polypeptide-9. Preferably this substance has PM 19,200 dalton. This platelet-like molecule plays an important role in the control of perifollicular vasculature during the hair cycle. Its production increases at the follicular keratinocytes level of the outer sheath of the follicular root during the anagen phase. This substance promotes the proliferation and migration of endothelial cells. It also increases the permeability and diameter of the capillaries during the anagen phase; in this way the growth of the hair is stimulated by the greater quantity of nutrients that flow to the follicle thanks to the development of the capillary network.

An additional component is represented by Synthetic Human Epidermal Growth Factor, INCI Name sh-Oligopeptide-1. Preferably this substance has PM 6,200 dalton. This component is a platelet-like molecule similar to mitogenic activity for keratinocytes and fibroblasts. It has been observed that this molecule inhibits the entry of the follicle in the catagen phase, activating the anagen and the growth of the hair. It is also important in controlling the orientation and elongation of the follicle due to its stimulating effect on the proliferation of basal keratinocytes and the outer sheath of the root. During the hair growth cycle acts as a switch to control the entry and exit from the anagen phase of the follicle.

According to certain embodiments,

the sh-Polypeptide-11 component can be present in a quantity ranging from 0.000001 to 20% by weight, more preferably from 0.001 to 10% even more preferably from 0.1 to 5% by weight;
the sh-Polypeptide-1 component may be present in a quantity ranging from 0.000001 to 20% by weight, more preferably from 0.001 to 10% even more preferably from 0.1 to 5% by weight;
the sh-Polypeptide-3 component may be present in a quantity ranging from 0.000001 to 20% by weight, more preferably from 0.001 to 10% even more preferably from 0.1 to 5% by weight;
the sh-oligopeptide-2 component can be present in a quantity ranging from 0.000001 to 20% by weight, more preferably from 0.001 to 10% even more preferably from 0.1 to 5% by weight;
the sh-Polypeptide-31 component can be present in a quantity ranging from 0.000001 to 20% by weight, more preferably from 0.001 to 10% even more preferably from 0.1 to 5% by weight;
the sh-oligopeptide-1 component may be present in an amount ranging from 0.000001 to 20% by weight, more preferably from 0.001 to 10% even more preferably from 0.1 to 5% by weight;
the sh-Polypeptide-9 component may be present in a quantity ranging from 0.000001 to 20% by weight, more preferably from 0.001 to 10% even more preferably from 0.1 to 5% by weight.

All percentages by weight previously described are to be understood with respect to the total weight of the composition.

The Applicant has found that the combination of the seven synthetic platelet-like previously described substances/molecules synergically stimulates the activity of the hair bulb, producing a stimulating effect of hair growth similar to that found using intradermal injections of PRP in the scalp.

According to another aspect, the present invention therefore relates to the cosmetic, non therapeutical method of treatment for the prevention and treatment of hair thinning, through a composition comprising sh-Polypeptide-11, sh-Polypeptide-1, sh-Polypeptide-3, sh-Oligopeptide-2, sh-Polipeptide-31, sh-Polypeptide-9, sh-Oligopeptide-1, and a cosmetically acceptable vehicle.

The composition of the invention is applied topically and is effective in preventing and/or treating the forms of thinning hair, thinning hair or even cases of baldness due for example to androgenetic alopecia or to telogenic defluvium.

The composition for the topical application of the invention may be present either in the liquid form as a lotion, solution, suspension or in the solid or semi-solid form such as gel, cream, ointment, balm, mask, transdermal release patch.

The composition of the invention can be formulated in a form suitable for trichological uses and for topical application, for example, as a solution or aqueous suspension, dispersion or gel.

In certain embodiments the vehicle of the aqueous composition is water-based and may contain an alcohol suitable for cosmetic applications such as ethyl alcohol and/or one or more cosmetically acceptable glycols such as propylene or pentylene or hexylene butylene glycol and mixtures thereof. Typically, the composition of the invention is in the form of lotion for trichological use.

In the form of aqueous based solution or suspension or dispersion the composition can generally contain about 1-99.9% of water. In some embodiments the water is present in a quantity comprised between 5-95%. In other embodiments the water is present in a quantity of 10-90% by weight.

In the case of the liquid formulation, the synergistic synthetic platelet-like molecules of the invention can be conveniently dissolved in a physiologically acceptable liquid vehicle such as water, alcohol, hydroalcoholic solution, glycerin and other suitable liquids for local application.

By way of example, the compositions of the invention in liquid form can be prepared by dissolving the water-soluble components in water and the remaining components in alcohol, to then combine the various fractions under stirring. The resulting mixture can then be buffered to reach a conveniently selected pH range of 5 to 7 to be compatible with the scalp pH and then be filtered and packaged in suitable containers such as flacons or vials.

In some embodiments the trichological compositions of the invention may comprise excipients commonly utilized in the formulation of local dermocosmetic preparations, such as preservatives, bactericidal agents, stabilizers, emulsifiers, surfactants, buffers, humectants, dyes and other excipients commonly utilized in the cosmetic preparation. The composition of the invention can be applied, in an effective quantity, directly on the scalp.

For example, in the treatment of androgenetic alopecia, a cosmetically active amount of lotion of the invention is applied directly to the scalp one or more times a day conveniently for cycles lasting 2-3 months alternated with rest periods.

According to some embodiments, the composition of the invention may also contain some amino acids such as cysteine and lysine and a glycoprotein to stimulate hair physiological growth, and can be utilized simultaneously, separately or sequentially to another dermocosmetic composition for trichological use to counteract hair loss.

For example, the other dermocosmetic composition may comprise one or more biologically active substances such as amino acids selected from hydroxyproline, aspartic acid and taurine, Caprylyl butyrate, Hydrolyzed Yeast Protein, Acetyl Tetrapeptide-3 and optionally one or more skin penetration enhancer for example pentylene glycol, decylene glycol, caprilyl glycol, as described in Swiss Patent No. 711466 in the name of the same Applicant. The researchers of the Applicant, starting from the previously described formulation, have further identified a combination of synthetic platelet-like molecules that found specific application in the dermocosmetic treatment of the tissues of the face and body.

In particular, the researchers found that by combining the seven platelet-like molecules previously described with further two similar platelet molecules such as sh-Polypeptide-8 and sh-Polypeptide-22, a mixture is obtained that has specific utilize in the cosmetic treatment of the skin and stimulates skin cell regeneration giving an effect of compactness and firming of the skin treated topically.

According to a second aspect, the present invention therefore provides a dermocosmetic composition comprising
Synthetic human acidic fibroblast growth factor or sh-Polypeptide-11
Synthetic human basic Fibroblast growth factor or sh-Polypeptide-1
Synthetic human keratinocyte Growth Factor or sh-Polypeptide-3
Synthetic human insulin like 1 Growth Factor or sh-Oligopeptide-2
Synthetic human insulin like 2 Growth factor sh-Polypeptide-31
Synthetic human vascular Endothelial Growth Factor or sh-Polypeptide-9
Synthetic human Epidermal Growth Factor or sh-Oligopeptide-1
Synthetic human Platelet-derived Growth Factor or sh-Polyeptide-8
Synthetic human Transforming Growth Factor beta-1 or sh-Polypeptide-22 and a cosmetically acceptable vehicle.

One of the components of the composition according to this last aspect of the invention is represented by Synthetic human PDGF having Inci Name: Sh-Polypeptide-8.

Preferably this substance has a PM 14.300 daltons.

Sh-Polypeptide-8 stimulates the collagen deposition in particular as a result of wounds and also stimulates the growth of epidermal cells. The Applicant observed that its presence within the composition improves skin elasticity by strengthening the structure of collagen and elastin.

The other component of the composition is represented by Synthetic Human TGF beta 1 having Inci Name sh-Polypeptyde-22. This substance preferably has a 12.900 dalton PM. The Applicant found that the presence of this substance inside the composition helps to slow down skin aging and promotes cell differentiation. Furthermore, this substance improves skin elasticity by strengthening the structure of collagen and elastin and stimulates the synthesis of collagen by providing an anti-wrinkle activity.

According to certain embodiments, the sh-Polypeptide-8 component can be present in a quantity ranging from 0.000001 to 20% by weight, more preferably from 0.001 to 10% even more preferably from 0.1 to 5% by weight; the sh-Polypeptide-22 component may be present in a quantity ranging from 0.000001 to 20% by weight, more preferably from 0.001 to 10% even more preferably from 0.1 to 5% by weight.

According to another aspect, the invention relates to the cosmetic method of treatment for the prevention and treatment of signs of skin aging such as facial corrugations, wrinkles, epidermis structural failures and skin laxity, through a composition comprising sh-Polypeptide-11, sh-Polypeptide-1, sh-Polypeptide-3, sh-Oligopeptide-2, sh-Polipeptide-31, sh-Polypeptide-9, sh-Oligopeptide-1, sh-Polypeptide-8, sh-Polypeptide-22 and a cosmetically acceptable vehicle.

In particular, the external application of a cosmetically effective amount of the composition of the invention increases the brightness of the treated areas and reduces the depth of wrinkles.

Typically, the molecular weights of the synthetic platelet-like substances or molecules described herein are expressed as weight average molecular weight (MW) (Da). Specifically, the average molecular weight of the hyaluronic acid mentioned in the application may be determined using standard methods in the field such as those which are disclosed in Ueno et al., 1988, Chem Pharm Bull. 36, 4971-4975; Wyatt 1993, Anal Chim Acta 272: 1-40; Watt Technologies 1999 “Light scattering University Dawn Course Manual and “Dawn Eos Manual” Wyatt Technology Corp. Santa Barbara Calif. (USA).

Typically, the composition of the invention is used for topical use by application on the skin of the face and/or body in need of treatment.

According to an embodiment the cosmetic composition according to these aspects of the invention can be in the form of hydrophilic gel, in semi-solid form as emulsion both oil in water and water in oil, of serum, lipophilic gel, hydrophilic or oily make-up removers, in stick shape for application, for example, on the lips, in the form of trans-dermal patches or make-up.

Alternatively, the cosmetic composition may be in liquid form, for example in the form of a lotion such as hydrophilic, hydroalcoholic lotions, or as cosmetic milks, oleolites, shampoos, baths foam, dispersions, suspensions, solutions that can be applied directly or by spray.

According to another embodiment, the cosmetic composition further comprises one or more of thickeners, solubilizers, preservatives, water, alcohols, glycerine, stabilizers, antioxidants and antibacterials cosmetically acceptable in quantities in line with those expected for the common cosmetic formulations.

In the composition of the invention, excipients typically utilized in the basic formulation of cosmetic products such as oils, glycerine, emollients, emulsifiers, dispersants in amounts typical for cosmetic compositions can be incorporated.

In the case of formulations in the form of solution, suspension, lotion, the water is present as a diluent or solvent, optionally mixed with other liquids used for the formulation of cosmetic compositions such as alcohols, such as ethyl alcohol, and glycols such as propylene glycol.

In the formulation of the cosmetic composition of the invention there may be other active substances such as vitamins, in particular vitamin A, vitamin E and their derivatives or other cosmetically active substances such as lecithin, hyaluronic acid, Palmitoyl Tripeptide-38, Acetyl Hexapeptide-37, Hydrolyzed Glycosaminoglycans and enhancer of skin penetration for example pentylene glycol, decylene glycol, caprilyl glycol.

The following examples are provided for the mere illustrative purpose of the present invention and they not be understood as limiting the scope of protection as is clear from the enclosed claims.

### Example 1

Composition for trichological use in the form of a lotion to stimulate hair growth containing 7 synthetic PS molecules and having the following formulation:

| Component | %(w/w) |
|---|---|
| ALCOHOL DENAT. | 0.1-99 |
| WATER | q.b. 100 |
| GLYCERIN | 0.1-10 |
| PENTYLENE GLYCOL | 0.00001-10 |
| CAPRYLYL GLYCOL | 0.00001-10 |
| DECYLENE GLYCOL | 0.00001-10 |
| BENZYL NICOTINATE | 0.0001-10 |
| SH-OLIGOPEPTIDE-1 | 0.000001-20 |
| SH-OLIGOPEPTIDE-2 | 0.000001-20 |
| SH-POLYPEPTIDE-1 | 0.000001-20 |
| SH-POLYPEPTIDE-11 | 0.000001-20 |
| SH-POLYPEPTIDE-3 | 0.000001-20 |
| SH-POLYPEPTIDE-31 | 0.000001-20 |
| SH-POLYPEPTIDE-9 | 0.000001-20 |
| DISODIUM EDTA | 0.0001-1 |
| MENTHOL | 0.0001-1 |
| BENZOPHENONE-4 | 0.001-1 |
| ALLANTOIN | 0.001-10 |
| ZINC ACETYLMETHIONATE | 0.0001-10 |
| QUATERNIUM-52 | 0.0001-1 |
| SODIUM HYDROXIDE | 0.0001-10 |
| SILANEDIOL SALICYLATE | 0.0001-10 |
| GLUTAMINE | 0.0001-10 |
| BIOTIN | 0.0001-10 |
| PHOSPHATIDYLCHOLINE | 0.0001-10 |

### Example 2

Composition in the form of a cream to reduce the signs of aging to be applied to the skin, containing 9 synthetic PS molecules and having the following formulation:

| Component | %(w/w) |
|---|---|
| WATER | q.b. 100 |
| DISODIUM EDTA | 0.001-1 |
| BETAINE | 0.1-5 |
| XANTHAN GUM | 0.01-10 |
| ACRYLATES/C10-30 ALKYL ACRYLATE | |
| CROSSPOLYMER | 0.1-10 |
| HYDROGENATED POLYDECENE | 0.1-10 |
| PRUNUS AMYGDALUS DULCIS OIL | 0.1-10 |
| TRITICUM VULGARE GERM OIL | 0.1-10 |
| DIMETHICONE | 0.1-10 |
| BUTYROSPERMUM PARKII BUTTER | 0.1-10 |
| ISOSTEARYL ISOSTEARATE | 0.1-10 |
| DIISOPROPYL DIMER DILINOLEATE | 0.1-10 |
| GLYCERYL STEARATE | 0.1-10 |
| CETEARYL ALCOHOL | 0.1-10 |
| STEARIC ACID | 0.1-10 |
| SODIUM LAUROYL GLUTAMATE | 0.1-10 |
| ROSA MOSCHATA SEED OIL | 0.1-10 |
| PHENOXYETHANOL | 0.001-1 |
| TOCOPHERYL ACETATE | 0.01-2 |
| DIAZOLIDINYL UREA | 0.001-1 |
| GLYCERIN | 0.001-10 |
| RETINYL PALMITATE | 0.0001-10 |
| SODIUM HYALURONATE | 0.00001-50 |
| HYALURONIC ACID | 0.00001-50 |
| HYDROLYZED SODIUM | |
| HYALURONATE | 0.00001-50 |
| HYDROLYZED HYALURONIC ACID | 0.00001-50 |
| HYDROLYZED | |
| GLYCOSAMINOGLYCANS | 0.00001-50 |
| CAPRYLYL GLYCOL | 0.00001-10 |
| PENTYLENE GLYCOL | 0.00001-10 |
| DECYLENE GLYCOL | 0.00001-10 |
| SH-OLIGOPEPTIDE-1 | 0.000001-20 |
| SH-OLIGOPEPTIDE-2 | 0.000001-20 |
| SH-POLYPEPTIDE-1 | 0.000001-20 |
| SH-POLYPEPTIDE-11 | 0.000001-20 |
| SH-POLYPEPTIDE-3 | 0.000001-20 |
| SH-POLYPEPTIDE-31 | 0.000001-20 |
| SH-POLYPEPTIDE-9 | 0.000001-20 |
| SH-POLYEPTIDE-8 | 0.000001-20 |
| SH-POLYPEPTIDE-22 | 0.000001-10 |

### Example 3

Composition in gel form to reduce the signs of aging to be applied to the skin, containing 9 synthetic PS molecules and having the following formulation:

| Component | %(w/w) |
|---|---|
| WATER | q.b. 100 |
| DISODIUM EDTA | 0.001-1 |
| CAPRYLYL GLYCOL | 0.00001-10 |
| PENTYLENE GLYCOL | 0.00001-10 |
| DECYLENE GLYCOL | 0.00001-10 |
| SH-OLIGOPEPTIDE-1 | 0.000001-20 |
| SH-OLIGOPEPTIDE-2 | 0.000001-20 |
| SH-POLYPEPTIDE-1 | 0.000001-20 |
| SH-POLYPEPTIDE-11 | 0.000001-20 |
| SH-POLYPEPTIDE-3 | 0.000001-20 |
| SH-POLYPEPTIDE-31 | 0.000001-20 |
| SH-POLYPEPTIDE-9 | 0.000001-20 |
| SH-POLYEPTIDE-8 | 0.000001-20 |
| SH-POLYPEPTIDE-22 | 0.000001-20 |
| TROPOLONE | 0.001-1 |
| GLYCERIN | 0.001-5 |
| PALMITOYL TRIPEPTIDE-38 | 0.00001-1 |
| ACETYL HEXAPEPTIDE-37 | 0.00001-1 |
| SODIUM HYALURONATE | 0.00001-50 |
| HYALURONIC ACID | 0.00001-50 |
| HYDROLYZED GLYCOSAMINOGLYCANS | 0.00001-50 |
| HYDROLYZED SODIUM HYALURONATE | 0.00001-50 |
| HYDROLYZED HYALURONIC ACID | 0.00001-50 |
| CARBOMER | 0.001-10 |

### Example 4

Activity test of the mixture of the 7 synthetic platelet-like molecules.

To demonstrate the regenerative efficacy of the complex based on synthetic platelet-like molecules, the Applicant has performed an evaluation of the increase in protein synthesis and cell proliferation by an in vitro test on culture of human keratinocytes (HaCaT).

The cells were cultured in DMEM Low Glucose containing 10% FBS and antibiotics. The cells were seeded in 24-well plates and left to grow for 24 h at 37° C. and 5% CO₂. Then they were deprived of the serum 6 hours before the experiment for cell cycle synchronization. After 6 hours, fresh culture medium was added without serum but containing the sample to be tested in order to reach different final dilutions. The sample was dissolved directly in the culture medium. The test was conducted in triplicate.

After 24 hours of exposure, the efficacy of the composition of 7 synthetic platelet-like molecules in the stimulation of cell proliferation of keratinocytes was determined. Untreated cells were utilized as a negative control, while cells treated with human insulin were utilized as a positive control.

The experiment showed an increase in the proliferation of the cells responsible for the synthesis of keratin of 34.5% compared to untreated cells.

To quantify proteins, a standard albumin calibration curve is constructed, and sample concentrations are calculated using the interpolation curve formula and relative absorbances. The averages of the individual data are then calculated, and for each sample the percentage of increase in protein synthesis is calculated, with respect to the negative control. The result was the following: % increase in protein synthesis in the culture treated with the 7 Synthetic Platelet-like Molecules=(μg/ml sample protein/μg/ml CN)*100 was 37% compared to untreated cells.

| MPS mix | Cell proliferation | protein synthesis |
|---|---|---|
| sh-Polypeptide-11 | +34.5% | +37% |
| sh-Polypeptide-1 | compared to the | compared to the |
| sh-Polypeptide-3 | untreated control | untreated control |
| sh-Oligopeptide-2 | | |
| sh-Polipeptide-31 | | |
| sh-Polypeptide-9 | | |
| sh-Oligopeptide-1 | | |

The increase in keratin production and in the cellular population of keratinocytes is significant of the effect on skin regeneration and/or hair growth.

Of the seven synthetic-like molecules of synthesis the Applicant has detected transdermal penetration by the use of Franz Cells, in the presence of 3 chemical enhancers of the combination of which the Swiss patent No. 711466 has been granted. The penetration data of the 7 synthetic platelet-like molecules of said experiment are reported to follow:

| | sh- | sh- | sh- | sh- | sh- | sh- | sh- |
|---|---|---|---|---|---|---|---|
| | Polypeptide- | Polypeptide- | Polyepetide- | Oligopeptide- | Polypeptide- | Polypetide- | Oligopeptide- |
| | 11 | 1 | 3 | 2 | 31 | 9 | 1 |
| molecular weight | 17.200 | 17.200 | 22.500 | 7.600 | 7.600 | 19.200 | 6.200 |
| in dalton | | | | | | | |
| % penetration | 91.4% | 90.8% | 91.4% | 95.8% | 93.2% | 88.3% | 96.4% |
| into the | | | | | | | |
| epidermis + | | | | | | | |
| dermis | | | | | | | |

From the experiments conducted, a very high % penetration in ex-vivo emerges, close to or higher 90% of the 7 platelet-like molecules and therefore indicative of a very high in vivo cutaneous absorption potential, through the follicular hosts, through the whole surface of the scalp and through the entire skin surface. Transepidermal and transdermal penetration follows the stimulation activity on bulbs and follicles aimed at hair growth and transcutaneous penetration for the regeneration of facial and body tissues.

1. Topical dermocosmetic composition comprising a mixture of the following seven synthetic platelet-like molecules:
synthetic human acidic fibroblast growth factor;
synthetic human basic fibroblast growth factor;
synthetic human keratinocyte Growth factor;
synthetic human insulin like 1 Growth factor;
synthetic human insulin like 2 Growth factor;
synthetic human vascular Endothelial Growth factor;
synthetic human Epidermal Growth factor;
a cosmetically acceptable vehicle, and further comprising a skin penetration enhancer which is a mixture of pentylene glycol, decylene glycol and caprylyl glycol.

2. The topical dermocosmetic composition according to claim 1 comprising synthetic human acidic fibroblast growth factor in a quantity from 0.000001 to 20% by weight, synthetic human basic fibroblast growth factor in a quantity from 0.000001 to 20% by weight, synthetic human keratinocyte Growth factor in a quantity of 0.000001 to 20% by weight, synthetic human insulin like 1 Growth factor in a quantity of 0.000001 to 20% by weight, synthetic human insulin like 2 Growth factor in a quantity of 0.000001 to 20% by weight, synthetic human vascular Endothelial Growth factor in a quantity of from 0.000001 to 20% by weight, synthetic human Epidermal Growth factor in a quantity of from 0.000001 to 20% by weight.

3. The topical dermocosmetic composition according to claim 1 in the form of an aqueous solution or hydroalcoholic solution.

4. The topical dermocosmetic composition according to claim 1 further comprising a glycoprotein, an amino acid selected from the group consisting of cysteine, lysine, hydroxyproline, aspartic acid and taurine, or mixtures thereof.

5. The topical dermocosmetic composition according to claim 1 further comprising a Hydrolyzed Yeast Protein and Acetyl Tetrapeptide-3.

6. (canceled)

7. The topical dermocosmetic composition according to claim 1 further comprising a platelet-like synthetic molecules selected from the group consisting of synthetic human platelet-derived Growth Factor and synthetic human Transforming Growth Factor beta 1 and a mixture thereof.

8. The topical dermocosmetic composition according to claim 7 comprising synthetic human platelet-derived Growth Factor in a quantity from 0.000001 to 20% by weight and synthetic human Transforming Growth Factor beta 1 in a quantity from 0.000001 to 20% by weight.

9. The topical dermocosmetic composition according to claim 7 in the form of a cream or emulsion oil in water or water in oil or hydrophilic gel.

10. A cosmetic method of treatment for the prevention or treatment of signs of skin aging comprising topically applying a composition according to claim 1 on the skin of a subject.

11. The cosmetic method of treatment according to claim 10 for the prevention or treatment of the wrinkles or of the facial or body corrugations or stretch marks or sagging of the skin of a subject.

12. A cosmetic method of treatment to prevent or treat scalp thinning said method comprising the topical application of a composition according to claim 1 on the scalp of a subject.
